(19) United States
(12) Patent Application Publication
(10) Pub. No.: US 2017/0232150 A1
(43) Pub. Date: Aug. 17, 2017

(54) SKELETAL-HOLE PLUG FORMED BY HARDENED BONE CEMENT

(71) Applicant: JOY MEDICAL DEVICES CORPORATION, Kaohsiung (TW)

(72) Inventors: Jiin-Huey Chern LIN, Winnetka, IL (US); Chien-Ping JU, Kansas City, MO (US); BING-CHEN YANG, Kaohsiung (TW)

(21) Appl. No.: 15/351,588

(22) Filed: Nov. 15, 2016

(30) Foreign Application Priority Data
Feb. 15, 2016 (TW) 105202181

(51) Int. Cl. A61L 27/56; A61F 2/28; A61L 27/02

(52) U.S. Cl. CPC A61L 27/56; A61L 27/02; A61F 2/28; A61L 2430/02; A61L 2430/12; A61F 2002/2839

(57) ABSTRACT

A skeletal-hole plug comprising a hardened bone cement, which has a circular or polygonal column, and the column is a porous hardened bone cement with a porosity of 30% to 90% by volume, which can be used to rapidly fill the skeletal holes resulted from the removal of bone screws, or from the removal of teeth, and to induce rapid growth of osteocytes in the skeletal holes.

### FIELD OF THE INVENTION

The present invention relates to a skeletal-hole plug made of a hardened bone cement that can be used to fill the skeletal holes resulted from the removal of bone screws, or from the removal of teeth.

### BACKGROUND OF THE INVENTION

In the surgeries for treating bone fractures or performing spinal corrections, bone plates and bone screws are frequently used to secure the fractured parts or the vertebrae being treated, and then removed after the secured parts have healed. As for the skeletal holes created and left after the bone screws are removed, or the skeletal holes remaining after teeth are removed, the current method of treatment is to allow the holes to heal on their own. Even though some of the surgeons have attempted to promote healing by filling bone grafts or granules of hardened bone cement into the skeletal holes, it is difficult to succeed as there is a great frictional resistance between the bone graft or granules of hardened bone cement, which prevents the bone grafts or the granules from flowing in a narrow confined space. Furthermore, a proper tool for injecting bone grafts or granules of hardened bone cement with a great frictional resistance into skeletal holes is simply not available. As a result of the frictional resistance, the bone grafts or granules of hardened bone cement could not be transferred by using the general syringes or thin tubes. Beside these difficulties, there is also a risk if the bone grafts or the granules should be successfully filled in the skeletal holes. That is loose bone grafts or granules may fall into the medullary cavity (mallow cavity) and migrate to undesired sites, raising safety concerns.

### SUMMARY OF THE INVENTION

A primary object of the present invention is to provide a skeletal-hole plug that overcomes the aforesaid shortcomings of the prior art, which has a circular or polygonal column, and the column is a porous hardened bone cement with a porosity of 30% to 90% by volume.

Because the skeletal-hole plug of the present invention is a circular or polygonal column, it can rapidly fill the skeletal holes resulted from the removal of bone screws, or from the removal of teeth.

The skeletal-hole plug of the present invention uses a bone cement with high bioresorption rate, and has a high porosity of 30% to 90% by mass, thus it can induce relatively faster growth of osteocytes in said skeletal holes.

Preferably, said column has a porosity of 50% to 90% by volume.

Preferably, said column has one end tapering towards the other end.

Preferably, said column has a length of 5 mm to 150 mm, and a diameter of 2 mm to 15 mm.

Preferably, said column has a length of 10 mm to 100 mm, and a diameter of 3 mm to 10 mm.

Preferably, said hardened bone cement is selected from the group consisting of: calcium phosphate, calcium sulfate, calcium oxide, calcium carbonate, calcium hydroxide, calcium magnesium phosphate, hydroxyapatite, silica, and a mixture thereof.

Preferably, said column has a substantially uniform cross section and does not have an enlarged head at either end thereof.

Preferably, the skeletal-hole plug of the present invention is used to fill skeletal holes resulted from the removal of bone screws.

Preferably, the skeletal-hole plug of the present invention is used to fill skeletal holes resulted from the removal of teeth.

The present invention further discloses a method of filling a skeletal hole resulted from removal of a bone screw or from removal of a tooth comprising filling the skeletal hole with a skeletal-hole plug comprising a circular or polygonal column, wherein the column is a porous hardened bone cement with a porosity of 30% to 90% by volume.

Preferably, the column is entirely in the skeletal hole, and more preferably a major portion of the column is surrounded by a cortical bone or cancellous bone forming the skeletal hole.

### BRIEF DESCRIPTION OF DRAWINGS

FIG. 1 is a perspective view showing a skeletal-hole plug fabricated according to a preferred embodiment of the present invention.

FIG. 2 is an enlarged photograph showing the high porosity on a surface of the skeletal-hole plug from FIG. 1.

### DETAILED DESCRIPTION OF PREFERRED EMBODIMENTS

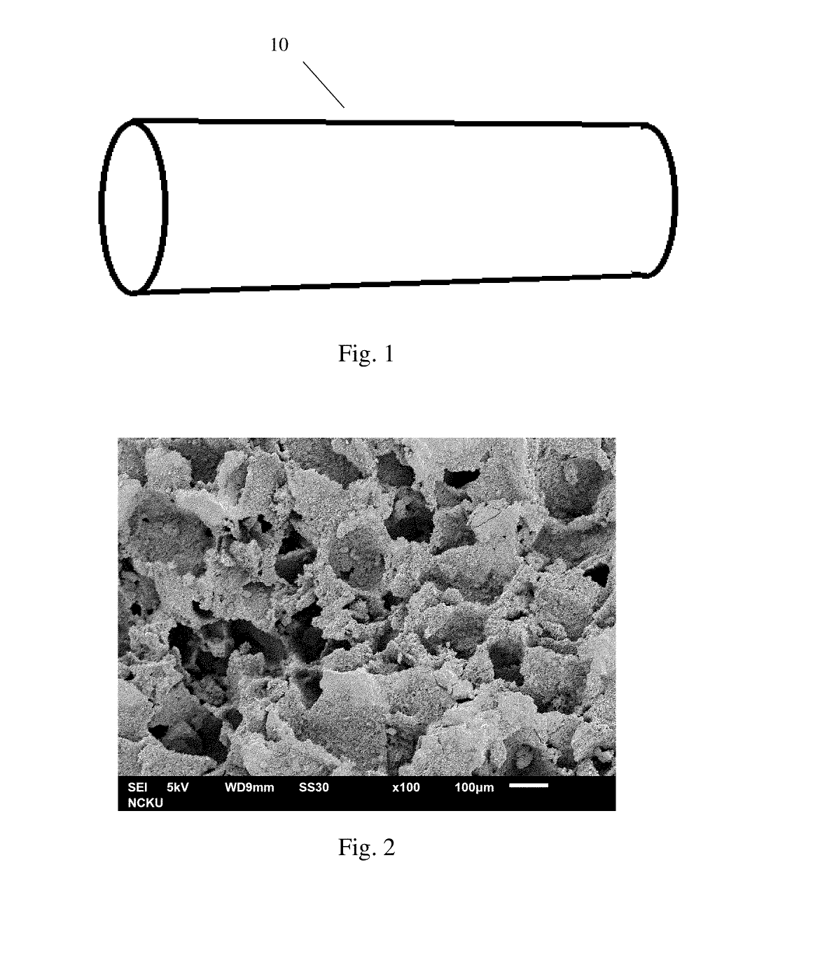

A skeletal-hole plug 10 fabricated according to a preferred embodiment of the present invention is shown in FIG. 1, which has a circular column. Said skeletal-hole plug 10 has a length of 80 mm, wherein one end thereof has a diameter of 8 mm, and another end thereof has a diameter of 6 mm. During usage, the relatively thinner end of said skeletal-hole plug is aligned with and inserted into a skeletal hole until all of it is inserted into the skeletal hole, or until the relatively thicker end of said skeletal-hole plug is almost levelled with or slightly protruding from the entry of the skeletal hole.

The shape and size of the skeletal-hole plug 10 of the present invention correspond to that of the bone screws used in current orthopedic surgeries, so surgeons can conveniently choose an appropriate skeletal-hole plug 10 to fill the skeletal holes resulted from the removal of bone screws, according to the shape and size of the removed bone screw.

Obviously, the shape of the skeletal-hole plug 10 disclosed in FIG. 1 can be slightly modified and still achieves the objectives of the present invention. For instance, the skeletal-hole plug may have an identical diameter disposed on both of the ends, and the edges of the two ends may be made with chamfers; the circular column may be modified to become a dodecagonal column, and the outer surface of the circular column may further have protruding edges (in axial direction or radial direction) or recesses disposed thereon.

A bone cement formula suitable for fabricating the skeletal-hole plug 10 of the present invention is based on calcium phosphates, such as the CPC (calcium phosphate cement) bone cement formula described in U.S. Pat. No. 4,612,053. The skeletal-hole plug 10 of the present invention may also use a bone cement formula comprising both calcium sulfates and calcium phosphates. For instance, a bone cement formula having a powder component and a setting liquid component is disclosed in U.S. Pat. No. 8,784,551 B2 by the inventors of the present invention, wherein the powder component includes a calcium sulfate source and a calcium phosphate source with a weight ratio of the calcium sulfate source less than 65%, based on the total weight of the calcium sulfate source and the calcium phosphate source, and the setting liquid component comprises ammonium ion (NH₄⁺) in a concentration of about 0.5 M to 4 M, wherein the calcium phosphate source includes tetracalcium phosphate (TTCP) and dicalcium phosphate in a molar ratio of TTCP to dicalcium phosphate of about 0.5 to about 2.5, and the calcium sulfate source is calcium sulfate hemihydrate (CSH), calcium sulfate dihydrate (CSD), or anhydrous calcium sulfate. The skeletal-hole plug 10 of the present invention may also use a bone cement formula based on calcium sulfate, as disclosed by the inventors of the present invention in US patent publication No. 2012/0093771 A1. The disclosures in the US Patents and patent publication mentioned above are incorporated herein by reference.

The bone cement formula used for fabricating the skeletal-hole plug 10 of the present invention further comprises a pore forming agent for forming pores in a hardened bone cement, wherein the hardened bone cement is immersed in an aqueous solution, so that the pore forming agent contained in the hardened bone cement is dissolved into the aqueous solution, thereby resulting in a porous hardened bone cement with high porosity.

A suitable pore forming agent includes can be selected from the group consisting of LiCl, KCl, NaCl, MgCl2, CaCl2, NaIO3, KI, Na3PO4, K3PO4, Na2CO3, amino acid-sodium salt, amino acid-potassium salt, glucose, polysaccharide, fatty acid-sodium salt, fatty acid-potassium salt, potassium bitartrate (KHC₄H₄O₆), potassium carbonate, potassium gluconate (KC₆H₁₁O₇), potassium-sodium tartrate (KNaC₄H₄O₆·4H₂O), potassium sulfate (K₂SO₄), sodium sulfate, sodium lactate and mannitol

A suitable process for preparing the skeletal-hole plug 10 of the present invention comprises: mixing together a powder component and a setting liquid component to form a bone cement paste; shaping said paste in a mold; removing the mold to form a dense block; immersing said dense block in an immersion liquid so as to allow the pore forming agent to become dissolved in said immersion liquid, creating pores in the dense block; and removing said block from said immersion liquid and drying the block to obtain a porous block of hardened bone cement. Preferably, said porous block of hardened bone cement has a porosity of 50-90% by volume, as shown in FIG. 2.

What is claimed is:

1. A skeletal-hole plug comprising a circular or polygonal column, and said column is a porous hardened bone cement having a porosity of 30% to 90% by volume.

2. The skeletal-hole plug of claim 1, wherein said column has a porosity of 50% to 90% by volume.

3. The skeletal-hole plug of claim 1, wherein said column has one end tapering towards the other end.

4. The skeletal-hole plug of claim 1, wherein said column has a length of 5 mm to 150 mm, and a diameter of 2 mm to 15 mm.

5. The skeletal-hole plug of claim 1, wherein said column has a length of 10 mm to 100 mm, and a diameter of 3 mm to 10 mm.

6. The skeletal-hole plug of claim 1, wherein said hardened bone cement is selected from the group consisting of: calcium phosphate, calcium sulfate, calcium oxide, calcium carbonate, calcium hydroxide, calcium magnesium phosphate, hydroxyapatite, silica, and a mixture thereof.

7. The skeletal-hole plug of claim 1, which is used to fill a skeletal hole resulted from the removal of a bone screw.

8. The skeletal-hole plug of claim 1, which is used to fill a skeletal hole resulted from the removal of a tooth.

9. A method of filling a skeletal hole resulted from removal of a bone screw or from removal of a tooth comprising filling the skeletal hole with a skeletal-hole plug comprising a circular or polygonal column, wherein the column is a porous hardened bone cement with a porosity of 30% to 90% by volume.

10. The method of claim 9, wherein the column is entirely in the skeletal hole.

11. The method of claim 10, wherein a major portion of the column is surrounded by a cortical bone or cancellous bone forming the skeletal hole

12. The method of claim 9, wherein said column has a porosity of 50% to 90% by volume.

13. The method of claim 9, wherein said column has one end tapering towards the other end.

14. The method of claim 9, wherein said column has a length of 5 mm to 150 mm, and a diameter of 2 mm to 15 mm.

15. The method of claim 9, wherein said column has a length of 10 mm to 100 mm, and a diameter of 3 mm to 10 mm.

16. The method of claim 9, wherein said hardened bone cement is selected from the group consisting of: calcium phosphate, calcium sulfate, calcium oxide, calcium carbonate, calcium hydroxide, calcium magnesium phosphate, hydroxyapatite, silica, and a mixture thereof.

17. The method of claim 9, wherein said skeletal hole is resulted from the removal of a bone screw.

18. The method of claim 9, wherein said skeletal hole is resulted from the removal of a tooth.

19. The skeletal-hole plug of claim 1, wherein said column has a substantially uniform cross section and does not have an enlarged head at either end thereof.

20. A skeletal-hole plug consisting of a circular or polygonal column, and said column is a porous hardened bone cement having a porosity of 30% to 90% by volume.
